(19) United States
(12) Patent Application Publication
(10) Pub. No.: US 2015/0232900 A1
(43) Pub. Date: Aug. 20, 2015

(54) PAPER FOLDING METHOD AND DEVICE FOR MANUFACTURING FILTER CARTRIDGES

(71) Applicant: MYKO TECH PRIVATE LIMITED, Dona Paula (IN)

(72) Inventors: Seshagiri Raghukumar, Dona Paula (IN); Hajib Naraharirao Madhavan, Dona Paula (IN); Jambulimgam Malathi, Dona Paula (IN)

(21) Appl. No.: 14/428,728

(22) PCT Filed: Sep. 17, 2013
(86) PCT No.: PCT/IB2013/058600

§ 371 Date: Mar. 17, 2015

(30) Foreign Application Priority Data
Sep. 18, 2012 (IN) 1744/MUM/2012

(51) Int. Cl. C12P 19/04

(52) U.S. Cl. CPC C12P 19/04

(57) ABSTRACT

This invention discloses a process of production of antiviral polysaccharides derived from extracellular culture filtrate of tharaustaochytrids, belonging to the marine protistan group of Labyrinthulomycetes. The antiviral polysaccharide shows a broad-spectrum antiviral activity, being active against viruses such as the enterovirus, retrovirus, adenovirus and cytomegalovirus.

### TECHNICAL FIELD OF THE INVENTION

The present invention relates to a process for the production of antiviral extracellular polysaccharides (EPS). More specifically the invention relates to the process of production of broad spectrum antiviral EPS from thraustochytrids, which are microorganisms belonging to the marine protists called Labyrinthulomycetes. The EPS is produced by growing thraustochytrids in culture media, harvesting the culture filtrate and extracting and purifying the extracellular polysaccharides. EPS obtained by this process are effective in treating one or more of several viruses, namely, enterovirus, cytomegalovirus, retrovirus and adenoviruses.

### BACKGROUND OF THE INVENTION

Viruses cause numerous serious diseases in human beings and animals. Antiviral vaccines provide protection against viral diseases. However, an already infected individual cannot be protected using vaccines and can be treated only by drugs. Unlike bacterial diseases, there are few antiviral drugs and just about 40 antiviral drugs are in use so far (Clercq, E. D. 2004. Antiviral drugs in current clinical use. Journal of Clinical Virology 30 (2004) 115-133). Most of these are against HIV and herpes viruses.

The four groups of viruses that are major cause of many kinds of diseases are enteroviruses, retroviruses, cytomegaloviruses and adenoviruses. Enteroviruses cause poliomyelitis, as well as infections resulting in a wide variety of symptoms ranging from mild respiratory illness (common cold), hand, foot and mouth disease, acute hemorrhagic conjunctivitis, aseptic meningitis, myocarditis, severe neonatal sepsis-like disease, and acute flaccid paralysis. No drugs are available to treat diseases caused by enteroviruses. Retroviruses are a group of enveloped RNA viruses that cause a variety of diseases. Among these, the Human Immunodeficiency Virus (HIV) that causes the Acquired Immunodeficiency Syndrome (AIDS) is a serious threat to human health. The nucleic acid derivative called 3′-azido-3′-deoxy thymidine (AZT) is the only available therapeutic agent for AIDS. A number of highly antiretroviral drugs control the disease, but have serious side effects (Ian McNicholl. 2011. Adverse Events of Antiretroviral Drugs”. University of California San Francisco.

http://hivinsite.ucsf.edu/InSite?page=ar-05-01). Cytomegalovirus or CMV belongs to the group of herpesviruses. Herpes viruses characteristically remain latent in the human body for prolonged periods. CMV infections are generally found in the salivary glands. The herpes, caused by Herpes Simplex Virus (HSV) establishes life long, latent infections in the peripheral nervous systems, escapes immune responses of the host and persists for a long time. It may also lie latent and may cause recurrent infections (Simmons A J. 2002. Clinical manifestations and treatment considerations of herpes simplex virus infection. Infect Dis 186: 71-77). Anti-herpes drugs include the nucleoside analogues. Acyclovir, valacyclovir, Famciclovir, Pencicloir, Cidofovir, Photoformatic Acid (PFA), also called foscarnet and Ara A or Vidarabine and Trifluorothymidine (TF) that block DNA replication. Of the above, acyclovir is licensed for the treatment of primary HSV infections. Development of resistant strains to acyclovir are known. The other drugs have problems such as low bioavailability, low potency, rapid degradation, toxicity and poor aqueous solubility. Adenovirus causes respiratory illness, as well as gastroenteritis, conjunctivitis, cystitis, and rash illness. There is, therefore, a dire need to look for more efficient antiviral drugs.

Marine organisms are potential sources of antiviral drugs (Yasuhara-Bell, J. and Y. Lu. 2010. Marine compounds and their antiviral activities. Antiviral Research 86 (2010) 231-240). Marine polysaccharides from various algae are useful as antiviral agents (K. Sogawa et al., 1998, Marine microalgal polysaccharides induces apoptosis in the human lymphoid cells. Journal of Marine Biotechnology 6; 35-38). Sulfated polysaccharides are potent and selective inhibitors of various enveloped viruses (Baba M, Snoeck R, Pauwels R, de Clercq E. 1988. Sulfated polysaccharides are potent and selective inhibitors of various enveloped viruses, including herpes simplex virus, cytomegalovirus, vesicular stomatitis virus, and human immunodeficiency virus. Antimicrob Agents Chemother 32, 1742-2745; Patent applications WO 2009027057 and WO 20090305). Others have shown antiviral activities, such as against Herpes Simplex Virus (HSV) (Huleihel M, Ishanu V, Tal J, Arad S: Antiviral effect of microalgal polysaccharides on Herpes simplex and Varicella zoster viruses. J. Appl. Phycol. 2001, 13:127-134), cell wall sulfated polysaccharide of the red microalga Porphyridium sp. against herpes simplex virus types 1 and 2 (HSV-1 and -2) (Huleihel M, Ishanu V, Tal J, Arad S M. 2002. Activity of Porphyridium sp. polysaccharide against herpes simplex viruses in vitro and in vivo. J Biochem Biophys Methods. 2002 50:189-200) are examples. Anti-HSV activity has been shown in a number of marine macroalgae (U.S. Pat. No. 5,089,481; Harden E A, Falshaw R, Carnachan S M, Kern E R, Prichard M N, 2009. Harden E A, Falshaw R, Carnachan S M, Kern E R, Prichard M N. Virucidal activity of polysaccharide extracts from four algal species against herpes simplex virus. Antiviral Research 83: 282-289). An inhibitor of enveloped virus replication, from the fresh water blue-green alga Spirulina platensis has been reported (Hayashi T, Hayashi M, Kojima I. A natural sulfated polysaccharide, calcium spirulan, isolated from Spirulina platensis: in vitro and ex vivo evaluation of anti-herpes simplex virus and anti-human immunodeficiency virus activities J Nat Prod 1996; 39:83-7). U.S. Pat. No. 5,089,481 of 1992 provides a pharmaceutical composition antiviral polysaccharide extracted from marine algae belonging to genera Nemacystus, Kjellmaniella, Laminaria, Undaria, Hizikia, Porphyra, Gelidium, Gloiopeltis, Gracilaria, Hemineura, Ulva, Spirogyra, Codium and Acetabularia, with retroviral activities. Red algal polysaccharides have been shown to inhibit HIV 1 and two different retroviruses in vitro (Talyshinsky M M, Y Y Souprun and M M Huleihel. 2002. Antiviral activity of red micro-algal polysaccharides against retroviruses. Cancer Cell International 2: 1-7). However, extraction from the cell wall of the algae is a cumbersome process.

EPS that are secreted outside cells are easier to extract and use EPS are common among microorganisms (Sutherland, I. W. 1996. Extracellular polysaccharides. In: Rhein, H. J. and Reed, G. (Eds.). Biotechnology. Vol. 6, VCH, Weinheim, pp. 615-627). Many microbial EPS, such as curdlan, gellan and xanthan are used in food and oil industry. The prime advantage of microbial EPS is that due to short life cycle of the microorganism, they can be produced in large amounts in short duration of time resulting in a highly economical and cost-effective production and downstream processes. Moreover, the EPS can be extracted in a pure form more easily. Therefore, the production of antiviral EPS using microorganisms grown in large volumes in fermenters and which secrete EPS has several advantages as the process is cost-effective, economical and production can be carried out at large scale, while being environmentally sustainable as well. The process would be further economical if the organism also produces yet another compound of industrial application. It would be even more useful if the EPS had broad-spectrum antiviral properties, since a single production process would suffice to treat several diseases.

Various bacteria and some marine algae produce antiviral, extracellular polysaccharides (Okutani K. 1992. Antiviral activities of sulfated derivates of a fucosamine containing polysaccharide of marine bacterial origin. Nippon Suisan Gakkaishi 58:927-30; S. Geresh and S. Arad, 1991. The Extracellular Polysaccharides of the Red Microalgae: Chemistry and Rheology Bio resource Technology 38: 195-201; T. Matsunaga et al., 1998, Applied Microbiology and Biotechnology 45: 24-27). EPS-1, a novel polysaccharide from the a strain of thermotolerant Bacillus licheniformis, isolated from a shallow marine hot spring of Vulcano Island (Italy) prevented HSV-2 replication in human peripheral blood mononuclear cells (PBMC) (Arena A, Maugeri T L, Pavone B, Iannello D, Gugliandolo C and Bisignano G. 2006. Antiviral and immunoregulatory effect of a novel exopolysaccharide from a marine thermotolerant Bacillus licheniformis. International Immunopharmacology 6: 8-13). EPS and their over sulfated derivatives of marine Pseudomonas showed antiviral activities against influenza virus Type A (Matsuda M, Shigeta S, Okutani K. 1999. Antiviral Activities of Marine Pseudomonas Polysaccharides and Their Oversulfated Derivatives. Marine Biotechnology 1: 68-73). EP0295961 describes EPS produced from the fungus species of Procaryomycetes, Eucaryomycetes, Ascomycetes and Basidiomycetes belonging to the families Ganodermeae, Tricholomateceae and Auriculariaceae have an inhibiting activity against adsorption of human immuno-deficiency virus (HIV) on cells as well as a function to inhibit an activity of the reverse transcriptase.

Among marine microbes that produce EPS, the heterotrophic group of marine single-celled protists, the thraustochytrids and aplanochytrids, belonging to the Labrinthulomycetes produce sulphated EPS, as well as the omega-3 poly unsaturated fatty acid (PUFA), docosahexaenoic acid (DHA) (Raghukumar, S. 2009. Thraustochytrid marine protists: production of PUFAs and other emerging technologies. Marine Biotechnology, 10: 631-640). Thraustochytrids are known to secrete polysaccharides externally into the culture medium (Jain, R., S. Raghukumar, R. Tharanathan and N. B. Bhosle. Extracellular polysaccharide production by thraustochytrid protists. Marine Biotechnology 7: 184-192) U.S. Pat. No. 8,232,090 describes the production of extracellular polysaccharides showing anti-hepatitis virus activity in a thraustochytrid strain of Schizochytrium limacinum MTCC 5249. However, this patent is restricted to the above strain and to hepatitis virus. Indian Patent Application 3257/MUM/2010 provides a process to produce an anti-herpes polysaccharide by the thraustochytrid strain of Schizochytrium limacinum MTCC 5249.

In view of the existing prior art technologies, there is an ongoing need for an improved production of antiviral polysaccharides from Labyrinthulomycetes for the treatment of herpes disease as well as capable to treat infections and diseases caused by enterovirus, retrovirus, adenovirus and cytomegalovirus.

### OBJECTS OF THE INVENTION

It is an object of the present invention to provide a process for the production of antiviral Extracellular polysaccharides (EPS) with broad-spectrum antiviral activities.

Yet another object of the present invention is to provide a process for the production of an antiviral EPS effective against enterovirus, retrovirus, cytomegalovirus and adenovirus.

It is still further object of the present invention to provide a process for the production of antiviral EPS from thraustochytrids and aplanochytrids, which are a subgroup of the marine protists belonging to the Labyrinthulomycetes.

### SUMMARY OF THE INVENTION

To achieve these and other objectives, the present invention provides a process of producing EPS with broad antiviral activities from Labyrinthulomycetes. The method of producing EPS from throustochytrids comprises multiple numbers of steps. It starts with culturing the cells in a culture medium along with a carbon source, nitrogen source, vitamin source and seawater of sodium salt for desired growth, then incubating the cells at a temperature in the range of 15-40° C. for a period of 3 to 8 days to obtain the culture, then separating the cell biomass from filtrate by means of methods well known in prior art, such as centrifugation and filtration, after that extracting the EPS from the said culture filtrates by using standard protocols, such as by using 70% isopropyl alcohol or by freezing the culture filtrate or combination thereof to precipitate the EPS and finally using the EPS in crude or purified form in antiviral preparations for topical or internal use against various viral diseases.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 shows a graph depicting effect of EPS from thraustochytrid strains MT-5 and MT-6 on viral plaques caused by enterovirus 71 in Vero cell lines in accordance to one or more embodiments of the invention.

FIG. 2 shows a graph depicting effect of EPS from thraustochytrid strains MT-5 and MT-6 on viral plaques caused by Human Adenovirus 5 in A549 cell line in accordance to one or more embodiments of the invention.

FIG. 3 shows a graph depicting effect of EPS from thraustochytrid strains MT-5 and MT-6 on viral foci caused by Retrovirus XMRV in PG54 cell line in accordance to one or more embodiments of the invention.

FIG. 4 shows a graph depicting effect of EPS from thraustochytrid strains MT-5 on viral plaques caused by Human Cytomegalovirus AD-169 in MRC5 cell lines in accordance to one or more embodiments of the invention.

### DETAILED DESCRIPTION OF THE INVENTION

In order to more clearly and concisely describe and point out the subject matter of the claimed invention, the following definitions are provided for specific terms which are used in the following written description.

By the term “Antiviral activity” we mean an agent that kills a virus or suppresses its ability to replicate and, hence, inhibit its capability to multiply and reproduce, as context requires.

By the term “Adenovirus” we mean a group of DNA-containing viruses that cause conjunctivitis and upper respiratory tract infections in humans, as context requires.

By the term “Cytomegalovirus” we mean a group a kind of herpesvirus that usually produces very mild symptoms in an infected person but may cause severe neurological damage in people, as context requires.

By the term “Extracellular polysaccharides” we mean a high molecular weight polymer that are composed of sugar residues and are secreted by a microorganism into the surrounding environment, as context requires.

By the term “Enterovirus” we mean a genus of positive sense single stranded RNA viruses associated with several human and mammalian diseases, as context requires.

By the term “in vitro” we mean production taking place in an artificial environment outside the living organism, as context requires.

By the term “in vivo” we mean production occurring within a living organism or in a natural setting, as context requires.

By the term “Protists” we mean a diverse group of eukaryotic microorganisms or colonial organisms with diverse nutritional and reproductive modes for example certain algae, as context requires.

By the term “Retrovirus” we mean a RNA virus that inserts a DNA copy of its genome into the host cell in order to replicate, e.g. HIV, as context requires.

The present invention overcomes the drawback of the existing technology by providing a process for production of EPS with broad range of antiviral activity from Labyrinthulomycetes belonging to thraustochytrids. The EPS obtained is effective against viruses such as the enterovirus, retrovirus, adenovirus and cytomegalovirus.

Any one of several cultures of microorganisms representative of thraustochytrids and aplanochytrids belonging to Labyrinthulomycetes, are grown individually in a culture medium containing seawater or sodium salts and suitable source of carbon and nitrogen for the desired growth and production of broad spectrum antiviral EPS. The carbon sources may include glucose, starch, glycerol and corn syrup, but are not limited to these. The nitrogen source may include ammonium, nitrate, urea, glutamate and peptone, but not restricted to these. The vitamin source may include yeast extract, corn steep liquor, beef extract, malt extract and soy extract or a defined vitamin mix, but not limited to these. The organism produces large molecular EPS in such a culture medium, which can be estimated by standard methods for polysaccharides. The organism is allowed to grow for a suitable duration of time in order for EPS to accumulate in the culture medium. This incubation period may last from 2 to 10 days and thereafter the cell biomass is separated from the culture filtrate by various means such as centrifugation and filtration. EPS present in the culture filtrate may be concentrated by passing the culture filtrate through an ultra filter of a suitable molecular cut-off size such that the EPS is retained in the concentrate. The concentrated EPS may be precipitated by various techniques known in prior art, such as the addition of 70% isopropyl alcohol, freezing the culture filtrate to precipitate the EPS and other methods, or a combination of these different methods. The precipitated water soluble EPS may then be centrifuged and later freeze-dried to yield a powder. The EPS may be further purified to obtain a pure fraction of the EPS that has the antiviral properties, by using a variety of techniques. Thus a solution containing the EPS may be separated through column chromatography or other standard methods to purify the active fraction from other fractions of polysaccharides if present and to remove impurities such that only the anti-viral portion of the EPS is obtained. Both the crude, as well as the purified EPS possess antiviral properties against a number of viruses, thus being a broad spectrum antiviral agent. The antiviral EPS may be used to treat a variety of viral infections including hepatitis viruses, herpes viruses, enteroviruses, retroviruses, cytomegalo viruses, adenoviruses, etc. The antiviral EPS may be used in a wide variety of formulations to treat viral infections. They may be incorporated in formulations for topical applications against certain viral infections. The purified antiviral EPS may be further fractionated to obtain the active moiety of the EPS that has antiviral activity The active moiety may be used as a drug for internal use in treating viral infections.

In order that this invention to be more fully understood the following preparative and testing examples are set forth. These examples are for the purpose of illustration only and are not to be construed as limiting the scope of the invention in any way.

### EXAMPLE 1

### Effect of EPS from Thraustochytrid Strains MT-5 and MT-6 on Viral Plaques Caused by Enterovirus

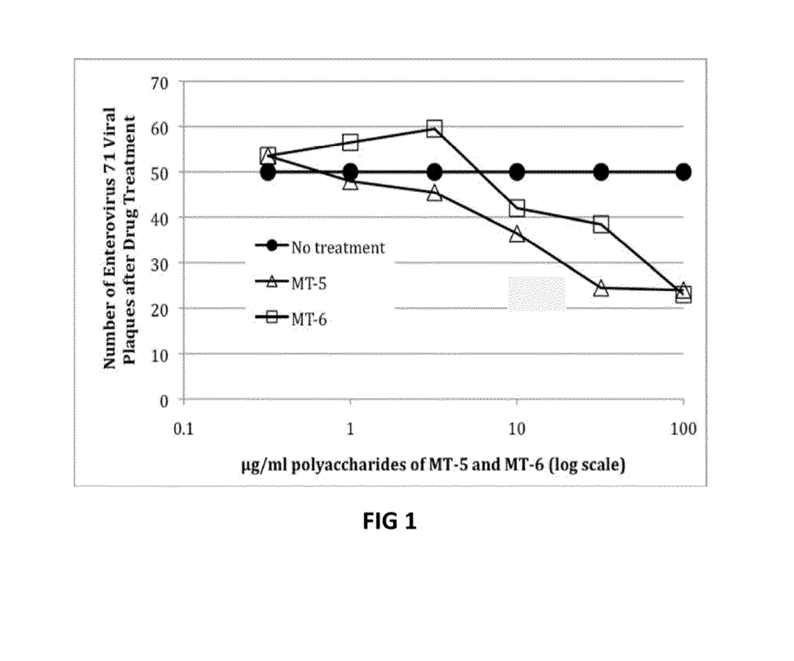

EPS from two thraustochytrids, designated MT-5 and MT-6 were tested for inhibition of Enterovirus. A stock solution of 20 mg/ml of the EPS was prepared by solubilizing in 10% DMSO for injection into the cell lines infected by the virus. Various dilutions of the stock EPS were prepared in the tissue culture medium, Dulbecco [symbol] Modified Eagle medium with 2% fetal bovine serum and antibiotics. The dilutions were: (1) 100 μg/ml; (2) 33 μg/ml; (3) 10 μg/ml; (4) 33 μg/ml; (5) 1.0 μg/ml; (6) 0.33 μg/ml. A plaque forming unit (PFU) inhibition assay in 6-well plates in vitro was carried out. A known concentration of cells from a Vero cell line was used to seed 6-well or 12-well plates for 24 hours. Each of the 6 different concentrations of the EPS was then added to duplicate wells and incubated for 1 hour and then approximately 50-100 plaque forming units of Enterovirus 71 in 10 [symbol] were added to each well. Virus was allowed to absorb to the cells for 2 hours. After the incubation the virus was removed and agarose solution with EPS at each different was added to the appropriate infected well. Plates were incubated in a 37° C. CO₂ incubator for 3 days. At the end of incubation, cultures were fixed and stained with crystal violet to visualize plaques. Plaques were counted and numbers recorded. The results are given in FIG. 1. Cells treated with EPS of both MT-5 and MT-6 showed a reduction in the number of viral plaques compared to cells not treated. MT-5 was effective at concentrations of 3.3 μg/ml and above, while MT-6 was effective at concentration of 10 μg/ml and above. The highest concentration of 100 μg/ml of both EPS resulted in more than a 50 reduction of viral plaques.

### EXAMPLE 2

### Effect of EPS from Thraustochytrid Strains MT-5 and MT-6 on Viral Plaques Caused by Human Adenovirus

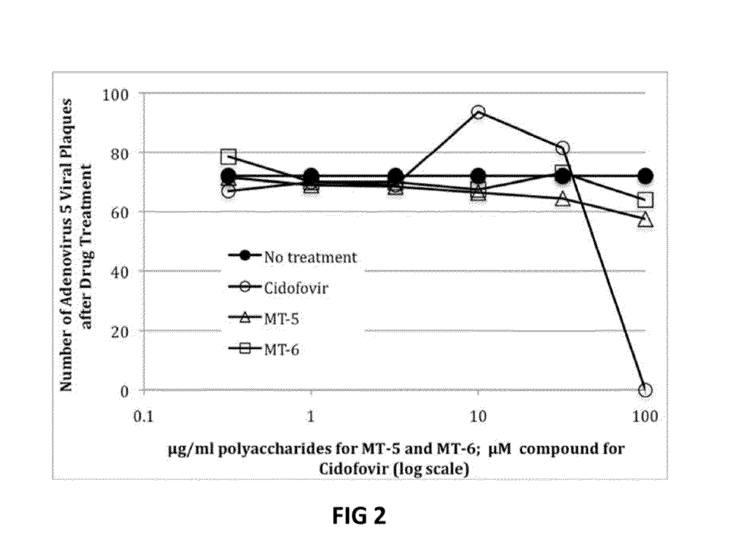

EPS from two thraustochytrids, designated MT-5 and MT-6 were tested for inhibition of Adenovirus. A stock solution of 20 mg/ml of the EPS was prepared by solubilizing in 10% DMSO for injection into the cell lines infected by the virus. Various dilutions of the stock EPS were prepared in the tissue culture medium, Dulbecco [symbol] Modified Eagle medium with 2% fetal bovine serum and antibiotics. The dilutions were: (1) 100 μg/ml; (2) 33 μg/ml; (3) 10 μg/ml; (4) 3.3 μg/ml; (5) 1.0 μg/ml; (6) 0.33 μg/ml. A plaque forming unit (PFU) inhibition assay in 6-well plates in vitro was carried out. A known concentration of cells from A549 cell line was used to seed 6-well or 12-well plates for 24 hours. Each of the 6 different concentrations of the EPS was then added to duplicate wells and incubated for 1 hour and then approximately 50-100 plaque forming units of Human Adenovirus 5 in 10 [symbol] were added to each well. Virus was allowed to absorb to the cells for 2 hours. After the incubation the virus was removed and agarose solution with EPS at each different was added to the appropriate infected well. Plates were incubated in a 37° C. CO₂ incubator for 5 days. The drug Cidofovir was kept as a positive control, at concentrations of 100, 33, 10, 3.3, 1.0 and 0.33 μM concentrations. At the end of incubation, cultures were fixed and stained with crystal violet to visualize plaques. Plaques were counted and numbers recorded. The results are given in FIG. 2. Cidofovir completely inhibited plaque formation at a concentration of 100 μM concentration but not less. Cells treated with EPS of both MT-5 and MT-6 showed a reduction in the number of viral plaques compared to cells not treated. MT-5 was effective at concentrations of 10.0 μg/ml and above, while MT-6 was effective at concentration of 100 μg/ml.

### EXAMPLE 3

### Effect of EPS from Thraustochytrid Strains MT-5 and MT-6 on Viral Foci Caused by Retrovirus

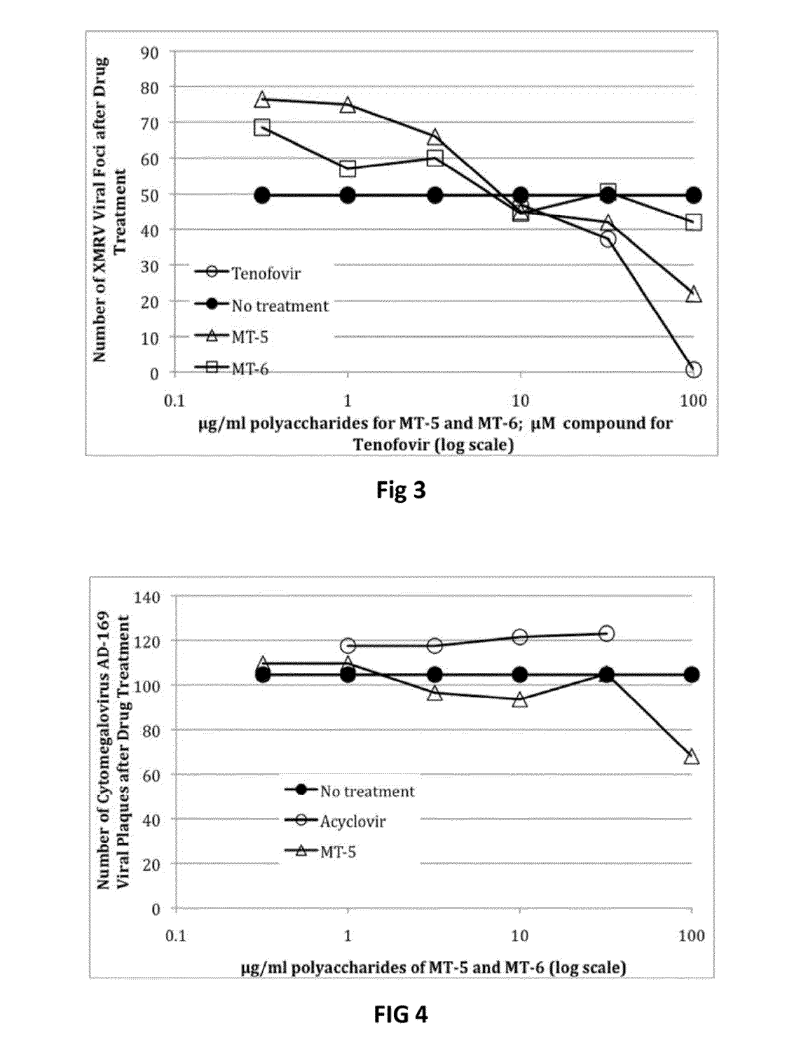

EPS from two thraustochytrids designated MT-5 and MT-6 were tested for inhibition of retrovirus A stock solution of 20 mg/ml of the EPS was prepared by solubilizing in 10% DMSO for injection into the cell lines infected by the virus. Various dilutions of the stock EPS were prepared in the tissue culture medium, Dulbecco [symbol] Modified Eagle medium with 2% fetal bovine serum and antibiotics. The dilutions were: (1) 100 [symbol] /ml; (2) 33 [symbol] /ml; 10 [symbol] /ml; (4) 3.3 μg/ml; (5) 1.0 μg/ml; (6) 0.33 μg/ml. A focus forming unit (FFU) inhibition assay in 6-well plates in vitro was carried out. A known concentration of cells from PG4 cell line was used to seed 6-well or 12-well plates for 24 hours. Each of the 6 different concentrations of the EPS was then added to duplicate wells and incubated for 1 hour and then approximately 50-100 focus forming units of the Retrovirus XMRV (Xenotropic Murine Leukemia Virus) in 10 [symbol] were added to each well. Virus was allowed to absorb to the cells for 2 hours. After the incubation the virus was removed and agarose solution with EPS at each different was added to the appropriate infected well Plates were incubated in a 37° C. CO₂ incubator for 5 days. The drug Tenofovir was kept as a positive control, at concentrations of 100, 33, 10, 3.3, 1.0 and 0.33 μM concentrations. Results are given in FIG. 3. At the end of incubation, cultures were fixed and stained with crystal violet to visualize the foci. Foci were counted and numbers recorded. The results are given in FIG. 3. Tenofovir was effective above concentrations of 10.0 [symbol] and completely inhibited focal formation at a concentration of 100 [symbol] concentration Cells treated with EPS of both MT-5 and MT-6 showed a reduction in the number of viral foci compared to cells not treated. MT-5 was effective at concentrations of 10.0 μg/ml and above, while MT-6 was effective at concentration of 100 μg/ml. The highest concentration of 100 μg/ml of EPS from MT-5 resulted in more than a 50% reduction of viral foci.

### EXAMPLE 4

### Effect of EPS from Thraustochytrid Strains MT-5 on Viral Plaques Caused by Human Cytomegalovirus

EPS from the thraustochytrid designated MT-5 were tested for inhibition of Cytomegalovirus. A stock solution of 20 mg/ml of the EPS was prepared by solubilizing in 10% DMSO for injection into the cell lines infected by the virus.

Various dilutions of the stock EPS were prepared in the tissue culture medium, Dulbecco [symbol] Modified Eagle medium with 2% fetal bovine serum and antibiotics. The dilutions were: (1) 100 [symbol] /ml; (2) 33 [symbol] /ml; (3) 10 [symbol] /ml; (4) 3.3 μg/ml; (5) 1.0 [symbol] /ml; (6) 0.33 μg/ml. A plaque forming unit (PFU) inhibition assay in 6-well plates in vitro was carried out. A known concentration of cells from MRC5 cell line was used to seed 6-well or 12-well plates for 24 hours. Each of the 6 different concentrations of the EPS was then added to duplicate wells and incubated for 1 hour and then approximately 50-100 plaque forming units of the Human Cytomegalovirus AD-169 in 10 [symbol] were added to each well. Virus was allowed to absorb to the cells for 2 hours. After the incubation the virus was removed and agarose solution with EPS at each different was added to the appropriate infected well. Plates were incubated in a 37° C. CO₂ incubator for 5 days. The drug Acyclovir was kept as a positive control, at concentrations of 100, 33, 10, 3.3, 1.0 and 0.33 μM concentrations. At the end of incubation, cultures were fixed and stained with crystal violet to visualize the plaques. Plaques were counted and numbers recorded. The results are given in FIG. 4. Acyclovir was not effective at any of the concentrations tested, up to a maximum of 100.0 μM. Cells treated with EPS of MT-5 showed a reduction in the number of viral plaques at a concentration of 100 [symbol] /ml. The EPS at this concentration resulted in more than a 35% reduction of viral plaques.

What is claimed is:

1. A process of producing extracellular polysaccharides (EPS) from thraustochytrid protists belonging to Labrinthulomycetes with broad range of antiviral activities wherein said process comprises:
a) culturing the cells in a culture medium comprising a carbon source, nitrogen source, vitamin source and seawater of sodium salt for desired growth;
b) incubating the cells at a temperature in the range of 15-40° C. for a period of 3 to 8 days to obtain the culture;
c) separating the cell biomass from filtrate by centrifugation and filtration;
d) extracting EPS from said culture filtrates by using 70% isopropyl alcohol or by freezing the culture filtrate or combination thereof to precipitate the EPS;
e) using said extracted EPS in crude or purified form in antiviral preparations for topical or internal use against various viral diseases.

2. The process as claimed in claim 1, wherein said EPS is active against enterovirus, retrovirus, adenovirus and cytomegalovirus.

3. The process as claimed in claim 1, wherein said carbon source is selected from a group consisting of glucose, starch, glycerol and corn syrup.

4. The process as claimed in claim 1, wherein said nitrogen source is selected from a group consisting of ammonium, nitrate, urea, glutamate and peptone.

5. The process as claimed in claim 1, wherein said vitamin source is selected from a group consisting of yeast extract, corn steep liquor, beef extract, malt extract and soy extract or a defined vitamin mix.

6. The process as claimed in claim 1, wherein said antiviral preparations prepared using EPS can be used to treat various infections and diseases caused by enterovirus, retrovirus, adenovirus and cytomegalovirus.
